(19) United States
(12) Patent Application Publication
(10) Pub. No.: US 2006/0287654 A1
(43) Pub. Date: Dec. 21, 2006

(54) Implant securing device and method

(75) Inventor: Jeffrey C. Posnick, Potomac, MD (US)

Correspondence Address: STEPTOE & JOHNSON LLP, 1330 CONNECTICUT AVENUE, N.W., WASHINGTON, DC, 20036, US

(73) Assignee: Jeffrey Posnick, Chevy Chase, MD (US)

(21) Appl. No.: 11/464,152

(22) Filed: Aug. 11, 2006

(51) Int. Cl. A61B 17/58

(52) U.S. Cl. 606/72

(57) ABSTRACT

An implant securing device includes a fixation component and a holding component. The fixation component can be secured to a portion of a cranial vault or other bone surface portion. The holding component can be secured to an implant, such as a precision cranial implant. The implant securing device can be used with an implant for reconstructing a defect of a cranial vault or orbital area of a mammal, such as a human.

### TECHNICAL FIELD

The present invention relates to the reconstruction of defects of the cranio-maxillofacial skeletal implants.

### BACKGROUND

The cranial vault region of a subject can present a bone defect, which can be reconstructed with a cranial implant.

Cosmetic and reconstructive cranio-maxillofacial implants are frequently manufactured from linear high-density polyethylene. The polyethylene implants can be porous to allow for tissue ingrowth. The implant shapes can be manufactured in a variety of shapes and volumes to augment or restore the contour of the cranio-maxillofacial skeleton, and to replace deficient soft tissue volume (e.g. ocular globe).

### SUMMARY

An implant securing device includes a fixation component configured to be secured to a portion of the bone surface adjacent to the defect and a holding component configured to be secured to a portion of an implant used to reconstruct the defect. The fixation component can include at least one position for attaching to a portion of a bone surface, and the holding component includes at least one flange. The implant securing device can be configured to fit a fixation element.

The implant securing device can include capture positions on the fixation component or the holding component. Capture positions can be positioned on opposing sides of a holding component and configured to receive a fixation element such as a nail, screw, rivet, sleeve or adhesive.

An implant securing device can include a holding component that includes a pin configured to stabilize an implant. For example, the holding component can form a sleeve with a superior flange and an inferior flange. A pin can be positioned between the superior and inferior flange to contact, pierce, or stabilize an implant.

The fixation element can be configured to fit at least one position for attaching to a portion of a bone surface in the fixation component. The fixation element can be a nail, screw, rivet, sleeve or adhesive. The position for attaching to a portion of a bone surface can be a hole or slot.

A first and second flange can be identical or different in size. A first and second position for attaching to a portion of a bone surface can be identical or different in size. A first and second flange can be configured to contact a superior and inferior edge of an implant, respectively.

In certain circumstances, an implant securing device can be configured to secure a precision implant having a contoured shape or an implant having a uniform height.

In another embodiment, a holding component can be configured to be positioned on an edge of an implant.

In another embodiment, the implant securing device can be configured to secure a customized surgical implant, a cranial implant, or an orbital implant.

In another embodiment, the implant securing device can be configured to secure an implant having least one nonporous or porous surface. The implant securing device or fixation element can include a metal, such as titanium, and can further include an additive or a coating.

In yet another embodiment, an implant securing device can have an aspect ratio from about 1:1 to 1:20.

A method of manufacturing an implant securing device can include obtaining material and molding a material to form a fixation component and a holding component.

A method of placing implant securing device into a mammal can include selecting a precision implant; selecting an implant securing device configured to secure the implant; attaching the implant securing device to the implant; and securing the implant securing device to a portion of a bone surface.

### DESCRIPTION OF DRAWINGS

FIG. 1 is a drawing depicting an implant used to reconstruct a skull defect. It is to be attached to a portion of the adjacent bone surface.

FIG. 2 is a drawing depicting an implant securing device.

FIG. 3 is a drawing depicting an implant securing device.

FIG. 4A is a drawing depicting an implant securing device.

FIG. 4B is a drawing depicting an implant securing device.

FIG. 4C is a drawing depicting an implant securing device.

FIG. 5A is a drawing depicting an implant securing device.

FIG. 5B is a drawing depicting an implant securing device.

FIG. 6A is a drawing depicting an implant securing device.

FIG. 6B is a drawing depicting an implant securing device.

### DETAILED DESCRIPTION

An implant securing device can include a fixation component and a holding component. The fixation component can be configured to be secured to a portion of a bone surface. The holding component can be configured to be secured to a portion of an implant. A fixation component can include at least one position for attaching to a portion of a bone surface, and the holding component can include at least one flange. The fixation component can also include two or more positions for attaching to a portion of a bone surface. The holding component can include two or more flanges.

The implant securing device can be configured to attach to a fixation element. The fixation element can be a nail, screw, rivet, sleeve or adhesive, for example. The fixation element can be configured to fit a position for attaching to a portion of a bone surface of a fixation component, thereby determining a position for attaching to a portion of a bone surface. The position for attaching to a portion of a bone surface can be a hole or a slot.

The implant securing device can include capture positions on the fixation component or the holding component. Capture positions such as holes, for example, can be positioned on opposing sides of a holding component and configured to receive a fixation element such as a nail, screw, rivet, sleeve or adhesive.

An implant securing device can include a holding component that includes a pin configured to stabilize an implant. For example, the holding component can form a sleeve with a superior flange and an inferior flange. A pin can be positioned between the superior and inferior flange to contact, pierce, or stabilize an implant.

The implant securing device can have a first and second flange at the holding component. The first and second flanges can be different in size or indentical in size. The implant securing device can have at least a first and second position for attaching to a portion of a bone surface. The first and second position for attaching to a portion of a bone surface can be different in size or identical in size.

The first and second flange can be configured to contact a superior edge of an implant, and in certain embodiments, an inferior edge of an implant. The implant securing device can be configured to secure a precision implant, such as an implant formed by molding polypropylene pellets to a contoured shape as taught by U.S. patent application Ser. No. 11/385,688, which is incorporated by reference herein. The implant securing device can be configured to secure a precision implant having a uniform height or a contoured shape. A uniform height can be a height of an implant that is substantially unchanged throughout the length of an implant. A contoured shape can be a shape that tapers toward at least one edge of an implant.

The implant securing device can include a holding component, which can be positioned along the edge of an implant. For example, at least one flange of a holding component can be configured to contact the edge of an implant, thereby securing the implant in place to prevent slippage or misalignment of the implant.

The implant can be a customized surgical implant, a cranial implant, or an orbital implant. The implant can include at least one nonporous or porous surface. The implant can include a metal. The metal can include titanium. The implant can include an additive or a coating.

The implant securing device can include a fixation component, a holding component, and can have an aspect ratio of about 1:1, 1:5, 1:10, or 1:20, for example. The implant securing device can have a tapered end. The implant securing device can have two or more configurations: a flat configuration, such as the configuration before an implant is inserted; and an open configuration after the implant has been inserted.

A method of manufacturing a cranial implant can include molding a material to form an implant securing device having a fixation component and a holding component. A material such as a metal or metal alloy can be obtained, heated to a softening temperature, and formed into a desired shape by bending or molding. Suitable molds are commercially available and include, but are not limited to, metal alloys of titanium and other materials known in the art. Specific molds can have varying heights and diameters.

A method of placing an implant securing device into a subject can include selecting an implant, such as a precision implant as described in U.S. patent application Ser. No. 11/385,688, which is incorporated by reference herein, selecting an implant securing device configured to secure the implant, attaching the implant securing device to the implant, and securing the implant securing device to a portion of a portion of a bone surface. An implant securing device can be configured to secure an implant based on it shape, size, adhesion, and desired location. Alternatively, the implant securing device can first be secured to a portion of a bone surface, and then the implant can be secured to the implant securing device.

An implant securing device can follow various patterns of attachment. For example, an implant securing device be secured to a portion of a bone surface, then an implant can be secured to the implant securing device, and then a second implant securing device can be secured to the implant, such that the implant is nestled between two or more implant securing device.

In another embodiment, two or more implant securing device can be secured to a portion of a bone surface, then an implant can be positioned between two or more implant securing device, which have been configured to hold the implant in place. A first implant securing device can be attached to a first location on a portion of a bone surface. A second implant securing device can be attached to a second location on a portion of a bone surface, such as a cranial vault surface.

In another embodiment, two or more implant securing device can be positioned opposite each other, proximate to each other, or adjacent to each other. Two or more flanges on the holding component can be the same or different in size. The flanges can be positioned opposite each other, proximate to each other, or adjacent to each other. Two or more holes in the fixation component can be the same or different in size. The holes can be positioned opposite each other, proximate to each other, or adjacent to each other.

A fixation component and holding component can be constructed of the same or different materials, and can have the same or different surface area, shape, flexibility, and elastic modulus. Either component can have any shape, for example, rectangular, square, rounded, circular, oval, trapezoidal, or an irregular shape. The shape can also be customized for each subject, taking into account the dimensions of the defect and the dimensions of the implant.

An implant can be a precision cranial implant which can be customized for each subject. The shape and size of the implant can be determined by the size and shape of the cranial defect, and the desired shape of the surface after surgery.

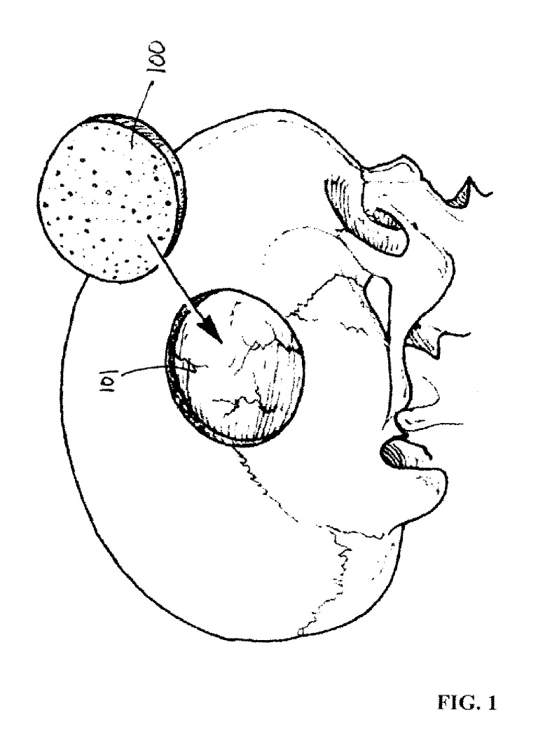

Referring to FIG. 1, an implant 100 can be used to reconstruct a defect such as a skull defect 101. The shape and size of an implant can be customized according to a subject's skull defect.

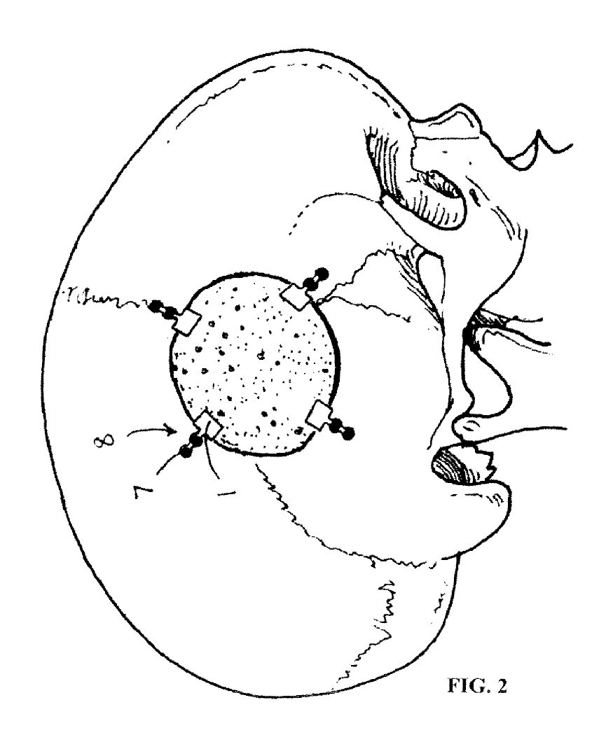

Referring to FIG. 2, an implant securing device 8 can include a fixation component 7 and a holding component 1. The fixation component can secure an implant to a portion of a bone surface adjacent to the defect. The holding component can secure an implant to the implant securing device.

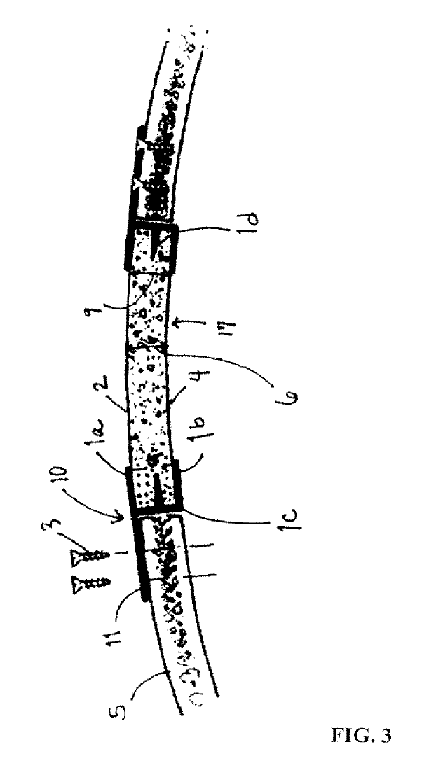

Referring to FIG. 3, an implant securing device 10 can include a fixation component 11, which can be secured to a portion of a bone surface adjacent to the defect 5 with a fixation element 3. A fixation element can be a screw, such as a titanium screw. The holding component can have at least one prong or flange 1a, 1b, which be secured to an implant 17. The implant securing device can have a sleeve component 1c, which can stabilize the implant and resist vertical movement, for example. The implant securing device can have a pin component 1d, which can pierce the implant and stabilize the implant and resist lateral or horizontal movement, for example. The implant can have a thickness 6. The thickness at the edge of the implant can determine the required distance between flanges of an implant securing device, and consequently, the size of the flange and the holding component. The holding component can have a first flange 1a, which can contact the superior surface 2 of an implant, and a second flange 1b, which can contact the inferior surface 4 of an implant. The flanges can be separated by a distance 9, according to the thickness at the edge of an implant.

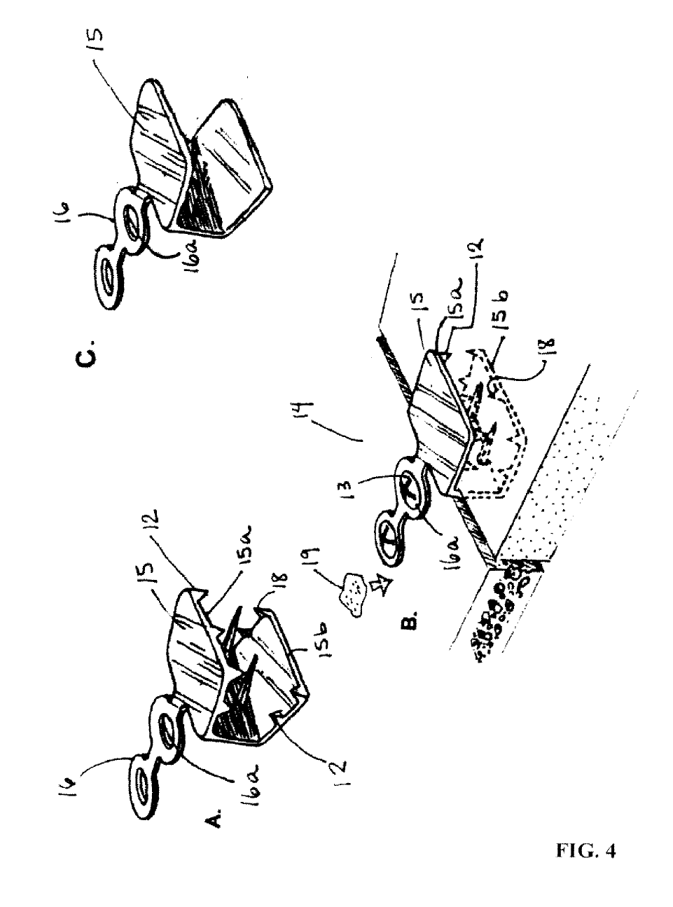

Referring to FIGS. 4A, 4B, and 4C, an implant securing device can include a holding component 15 and fixation component 16. The fixation component can include at least one position 16a for attaching to a portion of a bone surface 14 through which a fixation element 13, such as a screw or nail, can be affixed. An adhesive, such a biocompatible adhesive 19 can also be used as a fixation element. The implant securing device's holding component 15 can include a first flange 15a and a second flange 15b. Each flange can include minor flanges 12, 18, such as teeth or other protrusions, which can increase the surface area of a holding component, thereby enhancing attachment to an implant.

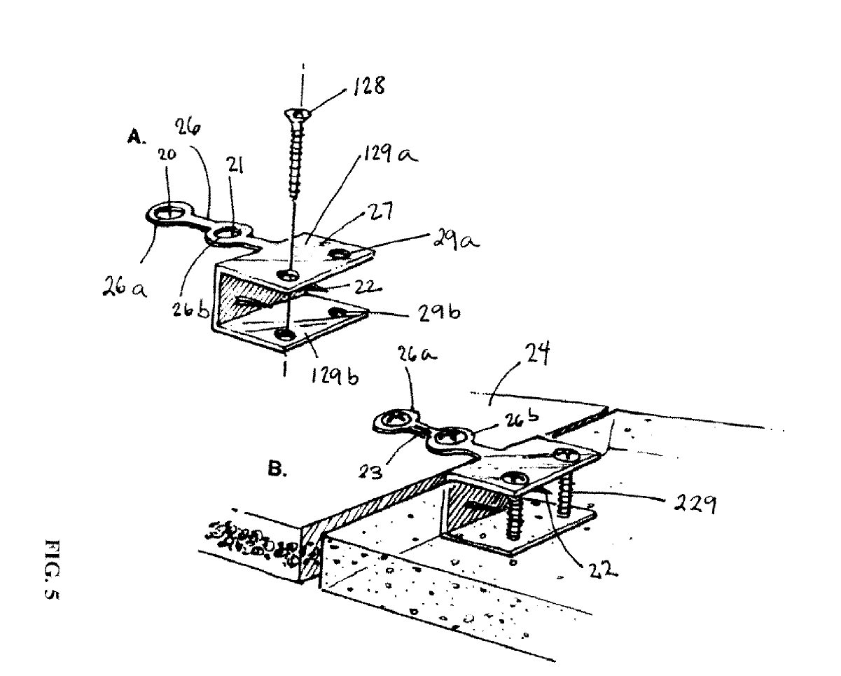

Referring to FIGS. 5A and 5B, an implant securing devices's fixation component 26 can include two or more positions 26a, 26b for attaching to a portion of a bone surface 24. An implant securing devices's holding component 27 can include two or more minor flanges 22. The positions for attaching can be a hole 20, 21 or a slot 23. The implant securing devices can be designed to have capture positions, such as holes, on opposing sides of the holding component. For example, a superior hole 29a can be positioned on a superior flange 129a and an inferior hole 29b can be positioned on an inferior flange 129b. A pair of opposing holes can capture a fixation element 128 such as a nail, screw, rivet, sleeve or adhesive, resulting in a sealed assembly 229.

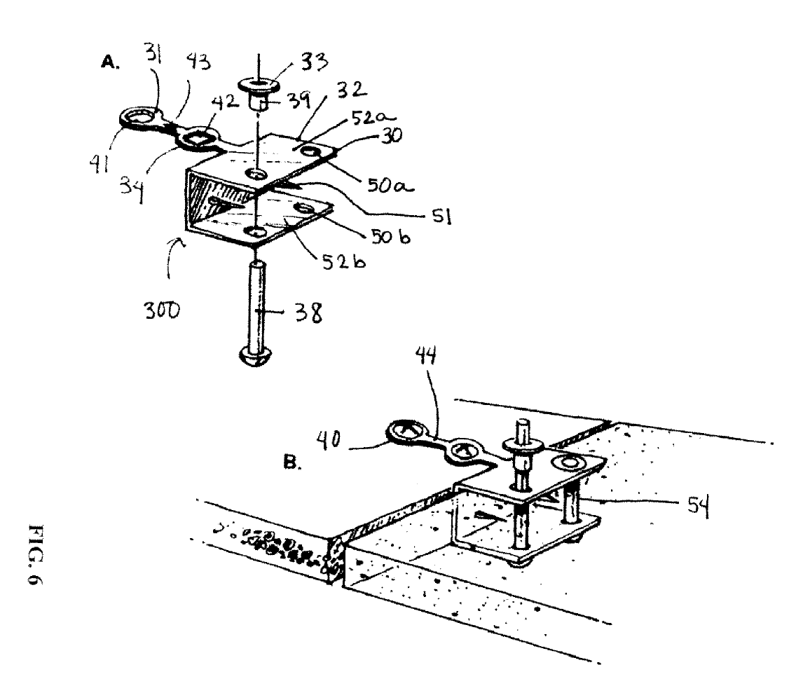

Referring to FIGS. 6A and 6B, an implant securing device 300 can be customized or designed to any desired shape, such as a rectangle, oval, or trapezoid. The implant securing device can have rounded edges 30 on either the fixation component 31 of the holding component 32. The implant securing device can have a position 34 for attaching to a portion of a bone surfaces customized or designed to any desired shape to suit any fixation element 33, such as a rivet 38, sleeve 39 or other fixation structure. For example, a position for attaching to a portion of a bone surface can be a rounded hole 41, an angled opening 42, a slot 43, or any combination thereof. The positions for attaching to a portion of a bone surfaces can be designed to be parallel to each other or staggered along the center portion 44 or edge 40 of a fixation component.

The implant securing device can be designed to have capture positions, such as holes, on opposing sides of the holding component. For example, a superior hole 50a can be positioned on a superior flange 52a and an inferior hole 50b can be positioned on an inferior flange 52b. A pair of opposing holes can capture a fixation element such as a nail, screw, rivet, sleeve or adhesive, resulting in a sealed assembly 54. Fixation elements can be captured through the fixation component or the holding component. An increased size or number of positions for attaching can increase the number of fixation points, which can promote stability of the implant securing device. An increased size or number of positions for attaching to a portion of a bone surfaces can also be designed to enhance the elastic modulus of the fixation component or holding component. Conversely, if a stiffer implant securing device is desired, a decreased size or number of openings can be chosen. Thus, the size and number of openings can be chosen depending on any number of factors, such as the size, shape, and material of the implant securing device, the desired flexibility of the implant securing device, the location of the implant securing device, and the type of fixation element, for example.

A cranial implant, such as a precision cranial implant can be composed of porous polypropylene and other materials. A cranial implant is described, for example, in U.S. patent application Ser. No. 11/385,688, which is incorporated by reference herein.

A cranial implant can be made of a polymeric material that is easily molded or shaped, resulting in a durable, porous and flexible material. In one embodiment, the cranial implant includes polypropylene pellets, which are then molded or fused into an implant of a desired shape and volume. The cranial implant can also include at least one functional additive that can confer additional properties, such as strength, flexibility, and biocompatibility, to enhance the implant's performance. The pellets can be shaped into a cranial implant by molding and fusing the pellets. In one embodiment, the pellets can be fused by sintering, for example.

A cranial implant can include a metal mesh, such as a titanium mesh. The mesh can be positioned on any surface of the cranial implant or in between polymeric layers of implant. The relative amounts of polymer and additive used can vary with the specific materials used, the desired strength and flexibility of the implant, and the properties conferred by a selected additive.

A cranial implant can be customized. For example, a customized implant can be designed based on 3-dimensional computed tomography (CT) scan models to make the implant patient-specific. CT or computer-aided design allows one to design a customized implant by obtaining information about the site of an implant (i.e. by scanning). Scanning can include using an MRI, an ultrasonic device, an x-ray machine, a camera, a scope, and combinations thereof to obtain information about the site of an implant. After information is obtained, one can process the information to generate information on the size and shape of the implant. After information is obtained and processed, one can transfer at least a portion of the generated information to a mold in order to form, at least partially, a custom implant from a moldable compound. A mold or a molding machine can include at least one mold cavity that can be varied in size or shape. The size or shape of the mold cavity can be adjusted or changed based at least partially on the data transferred to the mold or the molding machine, resulting in a customized shape. An example of using computer-aided design for prosthetic implants can be found in U.S. Pat. No. 6,786,930, which is hereby incorporated by reference.

The implant can also be customized by shaping, shaving, trimming, or burring the implant according to a desired shape. A burred shape refers to a sculpted or customized shape. The implant may also be modified according to the shape of the implant securing device, or vice-versa. The implant or implant securing device can be modified according to additional materials or grafts that may be involved in a surgical procedure.

A cranial implant can have a specific shape and aspect ratio, which renders it particularly suitable for implanting in cranio-maxillofacial areas. In one embodiment, a cranial implant can be designed to have a subtle “S” shape, which renders it suitable, for example, for augmenting or repairing the malar bone. The implant can have a main arc and at least one minor arc to the implant to follow the contour of a cranio-maxillofacial area. The implant can have at least one tapered edge. In one embodiment, the implant can be positioned over the zygomatic arch and adjacent to the infraorbital nerve. The subtle “S” shape of the implant can be designed to augment either the right or left side of the cranio-maxillofacial skeleton. The cranial implant can be a customized surgical implant, a chin implant, a cranial vault implant, an ear implant, a temporal implant, a mandibular angle implant, a paranasal implant, a nasal implant, a malar implant, an orbital implant, or an ocular globe implant. The cranial implant can be contoured or anatomical. For example, the malar implant can be shaped to augment, replace, or repair, the cheek and zygomatic areas of the cranio-maxillofacial skeleton. The ocular globe implant can be round or conical. The chin implant can be contoured or extended. The mandibular angle implant can be contoured, and the nasal and paranasal implants can have a crescent shape.

The implant can be molded to various heights and volumes within the specified aspect ratio. A cranial implant can have a total volume derived from a length, width, and height. A cranial implant may be molded to have an aspect ratio ranging from 1:3 to 1:20. For example, if the maximum height or thickness of the implant is 2 mm, the width of the implant can range from 6 to 40 mm. This range can allow a surgeon to select an implant that has the necessary durability and flexibility to augment, contour, or replace a specific cranio-maxillofacial area.

A cranial implant may be molded to have a uniform height, or a varying height. In one embodiment, a cranial implant can have a varying height, where the maximum height tapers to at least one edge of the implant. In another embodiment, a cranial implant can have a subtle “S” curve with a varying height, where the maximum height tapers to at least one edge of the implant.

An implant can also have a varying height and a substantially uniform porosity, thereby allowing even tissue ingrowth while effectively following the natural arch of the malar bone.

Other embodiments are within the scope of the following claims.

What is claimed is:

1. An implant securing device comprising a fixation component configured to be secured to a portion of a bone surface and a holding component configured to be secured to a portion of an implant.

2. The implant securing device of claim 1, wherein the fixation component includes at least one position for attaching to a portion of a bone surface, and the holding component includes at least one flange.

3. The implant securing device of claim 2, wherein the structure is configured to fit a fixation element.

4. The implant securing device of claim 3, wherein the fixation element is configured to fit at least one position for attaching to a portion of a bone surface in the fixation component.

5. The implant securing device of claim 1, wherein the structure includes capture positions on opposing sides of the holding component, the capture positions configured to receive a fixation element such as a nail, screw, rivet, sleeve or adhesive.

6. The implant securing device of claim 1, wherein the holding component includes a pin configured to stabilize an implant.

7. The implant securing device of claim 2, wherein the fixation element is a nail, screw, rivet, sleeve or adhesive.

8. The implant securing device of claim 2, wherein the position for attaching to a portion of a bone surface is a hole or slot.

9. The implant securing device of claim 2, wherein a first and second flange are different in size.

10. The implant securing device of claim 2, wherein a first and second position for attaching to a portion of a bone surface are different in size.

11. The implant securing device of claim 2, wherein a first and second flange are identical in size.

12. The implant securing device of claim 2, wherein a first and second position for attaching to a portion of a bone surface are identical in size.

13. The implant securing device of claim 1, wherein a first and second flange are configured to contact a superior and inferior edge of an implant, respectively.

14. The implant securing device of claim 1, wherein the implant securing device is configured to secure a precision implant having a contoured shape.

15. The implant securing device of claim 1, wherein the implant securing device is configured to secure a precision implant having a uniform height.

16. The implant securing device of claim 1, wherein holding component is configured to be positioned on an edge of an implant.

17. The implant securing device of claim 1, wherein the implant securing device is configured to secure a customized surgical implant.

18. The implant securing device of claim 1, wherein the implant securing device is configured to secure a cranial implant.

19. The implant securing device of claim 1, wherein the implant securing device is configured to secure an orbital implant.

20. The implant securing device of claim 1, wherein the implant securing device is configured to secure an implant having least one nonporous or porous surface.

21. The implant securing device of claim 1, wherein the implant securing device includes a metal.

22. The implant securing device of claim 21, wherein the metal includes titanium.

23. The implant securing device of claim 3, wherein the fixation element includes a metal.

24. The implant securing device of claim 1, further comprising an additive or a coating.

25. A implant securing device comprising a fixation component and a holding component and an aspect ratio from about 1:1 to 1:20.

26. The implant of claim 25, further comprising an additive or a coating.

27. The implant securing device of claim 25, wherein the structure includes capture positions on opposing sides of the holding component, the capture positions configured to receive a fixation element such as a nail, screw, rivet, sleeve or adhesive.

28. The implant securing device of claim 25, wherein the holding component includes a pin configured to stabilize an implant.

29. A method of manufacturing an implant securing device comprising obtaining a material and molding a material to form a fixation component and a holding component.

30. The method of claim 29, wherein the implant securing device includes a metal.

31. The method of claim 30, wherein the metal includes titanium.

32. The method of claim 29, further comprising placing a position for attaching to a portion of a bone surface in the fixation component.

33. The method of claim 32, wherein the position for attaching to a portion of a bone surface is a hole or slot.

34. The method of claim 29, wherein the structure includes capture positions on opposing sides of the holding component, the capture positions configured to receive a fixation element such as a nail, screw, rivet, sleeve or adhesive.

35. The method of claim 29, wherein the holding component includes a pin configured to stabilize an implant.

36. The method of claim 32, further comprising providing a fixation element configured to fit a position for attaching to a portion of a bone surface.

37. The method of claim 29, further comprising forming at least one flange on a holding component.

38. A method of placing implant securing device into a mammal comprising: selecting a precision implant; selecting an implant securing device configured to secure the implant; attaching the implant securing device to the implant; and securing the implant securing device to a portion of a bone surface.

39. The method of claim 38, wherein the structure includes capture positions on opposing sides of the holding component, the capture positions configured to receive a fixation element such as a nail, screw, rivet, sleeve or adhesive.

40. The method of claim 38, wherein the holding component includes a pin configured to stabilize an implant.

41. The method of claim 40, wherein the implant is a cranial implant.

42. The method of claim 40, wherein the implant is an orbital implant.

43. The method of claim 40, wherein the surface is a cranial vault surface.
